(19) United States
(12) Patent Application Publication
(10) Pub. No.: US 2006/0153835 A1
(43) Pub. Date: Jul. 13, 2006

(54) Treatment of pre-eclampsia in pregnant women using targeted apheresis

(76) Inventors: Henry J. Smith, Temecula, CA (US); James R. Smith, Laguna Niguel, CA (US)

Correspondence Address: STETINA BRUNDA GARRED & BRUCKER, 75 ENTERPRISE, SUITE 250, ALISO VIEJO, CA, 92656, US

(21) Appl. No.: 11/328,522

(22) Filed: Jan. 10, 2006

Related U.S. Application Data
(60) Provisional application No. 60/643,117, filed on Jan. 12, 2005.

(51) Int. Cl. A61K 39/395

(52) U.S. Cl. 424/133.1

(57) ABSTRACT

This invention uses “targeted apheresis” to treat pregnant women who are at risk of developing eclampsia. “Targeted Apheresis” is a process whereby the sFlt-1 receptors responsible for causing the disease symptoms are selectively removed from the blood by passing the blood through a cartridge containing either immobilized PIGF, and/or through a cartridge containing immobilized anti-sFlt-1 antibody. The sFlt-1 receptor is bound out and the cleaned blood is returned to the patient Removal of circulating sFlt-1 receptors will diminish the risk of developing eclampsia during pregnancy.

### CROSS-REFERENCE TO RELATED APPLICATIONS

This utility patent application claims priority to Provisional Patent Application Ser. No. 60/643,117, filed Jan. 12, 2005, entitled TREATMENT OF PRE-ECLAMPSIA IN PREGNANT WOMEN USING TARGETED APHERESIS.

### STATEMENT RE: FEDERALLY SPONSORED RESEARCH/DEVELOPMENT

Not Applicable

### BACKGROUND

Pre-eclampsia or toxemia during pregnancy is one of the leading causes of maternal and infant mortality. The symptoms of pre-eclampsia typically appear after the 20th week of pregnancy and are characterized by high blood pressure, edema and protein in the urine. In severe cases there is a massive rise in blood pressure that can result in severe complications, premature delivery of the baby and death of the mother or baby.

Pre-eclampsia can vary in severity from mild to life threatening. The mild form of pre-eclampsia is usually treated with bed rest and frequent monitoring. For moderate to severe cases, hospitalization is recommended and the patient is treated with blood pressure medication or anticonvulsant medications to prevent seizures. If the condition becomes life threatening to the mother or the baby the pregnancy is terminated and the baby is delivered pre-term.

Recent research has shown that the proper development of the fetus and the placenta appears to be mediated by several growth factors. One of these growth factors is placental growth factor (PlGF) and the other is vascular endothelial growth factor (VEGF). Placental growth factor (PlGF) is a VEGF family member that is capable of inducing proliferation, migration, and activation of endothelial cells. PlGF binds as a homodimer to the Flt-1 receptor found on trophoblast cells. VEGF is an endothelial cell-specific mitogen, an angiogenic inducer, and a mediator of vascular permeability. VEGF binds as a homodimer to the homologous tyrosine kinase receptors, the fms-like tyrosine kinase (Flt-1) receptor and the kinase domain receptor (KDR).

A soluble form of the Flt-1 receptor (sFlt-1) was recently identified. Circulating sFlt-1 receptors are believed to compete with the membrane fixed cellular Flt-1 receptors and act as a “physiologic sink” to down-regulate VEGF signaling pathways by binding to circulating PlGF and VEGF. It was postulated that women who produced large amounts of sFlt-1 early in their pregnancy were prone to develop pre-eclampsia.

Researchers have suggested several different therapeutic approaches to treat pre-eclampsia. One approach is to increase the level of PIGF and/or VEGF by injecting these compounds into the patient, or by utilizing drugs that stimulate the increased production of PIGF and/or VEGF. Increasing the amount of PlGF and VEGF in the presence of large amounts of sFlt-1 however, is analogous to driving a car and stepping on the gas while the brakes are still on. It would be preferable to reduce the level of circulating sFlt-1 so that the PlGF and VEGF can perform their functions.

One approach to inactivate the circulating sFlt-1 receptors is by injecting an anti-sFT-1 antibody into the patient. A difficulty with this approach is that any antibody that reacts with the active site of the sFlt-1 will also block the active site on the cellular Flt-1 receptor and may in fact exacerbate the problem.

It would be preferable to develop a more safe and effective process of reducing the level of circulating sFlt-1 receptors in order to allow the PlGF and VEGF to perform their functions.

This invention teaches a novel method of treating pre-eclampsia by reducing the circulating level of sFlt-1 using “targeted apheresis”.

### BRIEF SUMMARY

The main application of this invention is in the treatment of pregnant women who are at risk of developing eclampsia using a process of “targeted apheresis”. “Targeted Apheresis” is a process whereby only the sFlt-1 receptors responsible for causing the disease symptoms are selectively removed from the blood by passing the blood through a cartridge containing either immobilized PIGF and/or through a cartridge containing immobilized anti-sFlt-1 antibody. The sFlt-1 receptor is bound out by the targeted apheresis cartridge and the cleaned blood is returned to the patient Removal of circulating sFlt-1 receptors will diminish the risk of developing eclampsia during pregnancy.

### DETAILED DESCRIPTION

This invention teaches a method of targeted apheresis for treating pre-eclampsia during pregnancy. Targeted apheresis is used to remove the circulating sFlt-1 receptors that are believed to be responsible for the symptoms of eclampsia. The removal of sFlt-1 receptors can be achieved using two different types of targeted apheresis cartridge. One cartridge type utilizes immobilized anti-Flt-1 antibody and the other cartridge type utilizes immobilized PIGF.

Depending on the individualized circumstances patients may be treated with either one or both types of apheresis cartridge.

Typically, pregnant women who exhibit laboratory findings and clinical signs of developing pre-eclampsia are candidates for targeted apheresis. Treatment will consist of one or more targeted apheresis treatments performed during the risk period of the pregnancy. This will typically begin about the 20th week of pregnancy and continue on a periodic basis until delivery.

Targeted Apheresis Using Anti-Flt-1 Antibody

Preparation of the Immobilized Anti-Flt-1 Antibody Cartridge.

Antibody to Flt-1 receptor epitope(s) are produced according to standard laboratory methods. Laboratory animals are immunized with the antigen and the serum collected. The Flt-1 antibody is purified using standard laboratory methods including salt precipitation, gel-filtration, affinity chromatography and other purification methods. These and similar methods are known to those skilled in the art and are within the scope of this invention. The anti-Flt-1 antibody may be of the IgG class, or the IgM class, or the IgA class of immunoglobulin.

Alternatively, monoclonal antibody to Flt-1 receptor epitope(s) can be developed using standard laboratory methods to produce hybridomas. The monoclonal antibodies may be of the IgG class or of the IgM class of immunoglobulin, and they may be of murine origin or of human origin. These and similar methods of developing monoclonal antibodies are known to those skilled in the art and are within the scope of this invention.

The composition of the antibody used in the targeted apheresis device may be the whole antibody molecule or the binding fragment of the antibody molecule. In this invention the term “antibody” refers to the whole molecule and/or the binding site of the molecule.

The anti-Flt-1 antibodies are immobilized by chemically coupling them to an insoluble support matrix such as agarose beads. For example, agarose beads are activated using cyanogen bromide and the antibody protein is incubated with the activated agarose to allow coupling to occur. The unconjugated material is removed by washing with buffer and the antibody bound agarose is packed into the targeted apheresis device. There are many different methods of chemically coupling proteins to a variety of insoluble support matrixes. These matrix materials and methods of protein coupling are known to those skilled in the art and are within the scope of this invention.

Typically, the apheresis device will be constructed as a cylinder with an inlet to allow plasma to enter at one end, and an outlet at the opposite end to allow the cleaned plasma to exit and be returned to the patient. Other device configurations may also be designed and are within the scope of this invention. The cartridge device is constructed of material that is nontoxic and which provides rigid support to the agarose within. Typically, the material will of a plastic composition such as polystyrene, or polyvinyl, or polypropylene or other similar material. There is an inside filter at the bottom of the device to prevent the agarose beads from leaving the device. There is also an inside filter at the top of the device to contain the agarose within the device. Typically these filters are composed of plastic and/or cellulosic material and have pores that will allow thru passage of fluid such as plasma, but not particulate material such as agarose beads. The manufacture of these types of devices and the materials used are known to those skilled in the art and are within the scope of this invention.

Apheresis Procedure Using Immobilized Anti-Flt-1 Antibody

The overall procedure for targeted apheresis is the same as that used in conventional apheresis. Briefly, blood from the patient is circulated extra corporeally using standard apheresis equipment. The blood is separated into the cellular elements (red blood cells, white blood cells and platelets) and fluid (plasma) elements using differential centrifugation or a membrane filter. The plasma is then pumped through the targeted apheresis device where the anti-Flt-1 antibodies will bind to the circulating sFlt-1 receptors and remove them from circulation. The cleaned plasma is then mixed with the cellular blood elements and returned to the patient.

Targeted apheresis differs from conventional apheresis in that in targeted apheresis only the pathological elements responsible for the disease or disease symptoms are removed.

The targeted apheresis cartridge may be employed as a single use device or it may be regenerated and used multiple times. To regenerate the device an elution buffer solution is passed through the device to release the sFlt-1 bound to the immobilized antibody. For example, an elution buffer such as glycine-HCl buffer pH 2 will dissociate antigen:antibody bonds. The unbound antigen is washed out of the device and the regenerated antibody-agarose matrix is then washed and stored in physiological buffer such as phosphate buffered saline pH 7.2 with preservatives. Other similar eluting buffers and storage buffers are known to those skilled in the art and are within the scope of this invention. Typically, the cartridge device is stored in the cold at 2-8 C

Targeted Apheresis Using PIGF.

Preparation of the Immobilized PlGF Cartridge.

PlGF is expressed by cytotrophoblasts and syncytiotrophoblasts and secreted into the blood. PlGF can be isolated from blood using standard laboratory methods such as gel-filtration, high pressure liquid chromatography and affinity chromatography. These and other protein purification methods are known to those skilled in the art and are within the scope of this invention.

PlGF can also be prepared using genetic engineering methods. These procedures are known to those skilled in the art and are considered within the scope of the invention. For example, the genetic code for PlGF is cloned using the polymerase chain reaction and attached to plasmid DNA. The altered plasmid DNA is used to transform E. Coli bacteria which are grown in fermentation tanks. The transformed bacteria produce human PlGF which is purified using standard methods such as ion exchange, gel permeation and reverse-phase chromatography. Alternatively, the recombinant PlGF can be produced using other recombinant protein expression systems such as Spodoptera frugiperda insect cells without affecting the novelty of this invention. The recombinant PlGF may be expressed either complete, or as a fragment which has Flt-1 binding capacity, or as a fusion protein, without affecting the novelty of this invention. In this context, the term PlGF refers to the intact PlGF molecule and/or to the sFlt-1 receptor binding site of the PlGF molecule and/or to the Flt-1 receptor binding site of the PlGF molecule when it is a part of a recombinant fusion protein.

The PlGF is immobilized by chemically coupling it to an insoluble support matrix such as agarose beads. For example, agarose beads are activated using cyanogen bromide and the PlGF protein is incubated with the activated agarose to allow coupling to occur. The unconjugated material is removed by washing with buffer and the PlGF bound agarose is packed into the targeted apheresis device. There are many different methods of chemically coupling proteins to a variety of insoluble support matrixes. These matrix materials and methods of protein coupling are known to those skilled in the art and are within the scope of this invention.

Typically, the apheresis device will be constructed as a cylinder with an inlet to allow plasma to enter at one end, and an outlet at the opposite end to allow the cleaned plasma to exit and be returned to the patient. Other device configurations may also be designed and are within the scope of this invention. The cartridge device is constructed of material that is nontoxic and which provides rigid support to the agarose within. Typically, the material will of a plastic composition such as polystyrene, or polyvinyl, or polypropylene or other similar material. There is an inside filter at the bottom of the device to prevent the agarose beads from leaving the device. There is also an inside filter at the top of the device to contain the agarose within the device. Typically these filters are composed of plastic and/or cellulosic material and have pores that will allow thru passage of fluid such as plasma, but not particulate material such as agarose beads. The manufacture of these types of devices and the materials used are known to those skilled in the art and are within the scope of this invention.

Apheresis Procedure Using Immobilized PlGF

The overall procedure for targeted apheresis is the same as that used in conventional apheresis. Briefly, blood from the patient is circulated extra corporeally using standard apheresis equipment. The blood is separated into the cellular elements (red blood cells, white blood cells and platelets) and fluid (plasma) elements using differential centrifugation or a membrane filter. The plasma is then pumped through the targeted apheresis device where the circulating sFlt-1 receptors will bind to the immobilized PlGF and be removed from the circulation. The cleaned plasma is then mixed with the cellular blood elements and returned to the patient.

Targeted apheresis differs from conventional apheresis in that in targeted apheresis only the pathological elements responsible for the disease or disease symptoms are removed.

The targeted apheresis cartridge may be employed as a single use device or it may be regenerated and used multiple times. To regenerate the device an elution buffer solution is passed through the device to release the sFlt-1 bound to the immobilized PlGF. The released sFlt-1 receptors are washed out of the device and the regenerated PlGF-agarose matrix is then washed and stored in physiological buffer such as phosphate buffered saline pH 7.2 with preservatives. Other similar eluting buffers and storage buffers are known to those skilled in the art and are within the scope of this invention. Typically, the cartridge device is stored in the cold at 2-8 C

The above description is given by way of example, and not limitation. Given the above disclosure, one skilled in the art could devise variations that are within the scope and spirit of the invention disclosed herein. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A method of using targeted apheresis to treat pre-eclampsia in pregnant women.

2. A method according to claim 1 whereby the process of targeted apheresis utilizes a device containing immobilized anti-Flt-1 antibody.

3. A method according to claim 2 where the antibody is a polyclonal antibody.

4. A method according to claim 2 where the antibody is a monoclonal antibody.

5. A method according to claim 2 where the anti-Flt-1 antibody consists of the whole molecule or the binding fragment of the antibody molecule.

6. A method according to claim 2 where the antibody is conjugated to an agarose support matrix or similar support matrix.

7. A method according to claim 2 where the device is a disposable device for single use only.

8. A method according to claim 2 where the device is regenerated and used multiple times.

9. A method according to claim 1 whereby the process of targeted apheresis utilizes a device containing immobilized placental growth factor (PlGF).

10. A method according to claim 9 where the PlGF is isolated from blood.

11. A method according to claim 9 where the PlGF is a recombinant protein or part of a fusion recombinant protein.

12. A method according to claim 9 where the PlGF is conjugated to an agarose support matrix or similar support matrix.

13. A method according to claim 9 where the device is a disposable device for single use only.

14. A method according to claim 9 where the device is regenerated and used multiple times.
